(19) United States
(12) Patent Application Publication
(10) Pub. No.: US 2019/0265185 A1
(43) Pub. Date: Aug. 29, 2019

(54) NON-BIOHAZARDOUS SOLUTIONS AND METHODS FOR TESTING ANALYSERS

(71) Applicant: Universal Biosensors Pty Ltd, Rowville, Victoria (AU)

(72) Inventors: Ronald CHATELIER, Bayswater (AU); Peter Michael RONALD, Ashburton (AU)

(21) Appl. No.: 16/342,864

(22) PCT Filed: Oct. 20, 2017
(86) PCT No.: PCT/AU2017/051142

§ 371 Date: Apr. 17, 2019

Related U.S. Application Data
(60) Provisional application No. 62/410,565, filed on Oct. 20, 2016.

(51) Int. Cl. G01N 27/327; C12Q 1/56; G01N 27/49

(52) U.S. Cl. CPC G01N 27/3271; C12Q 1/56; C12Y 304/21005; C12Y 304/21004; G01N 27/49

(57) ABSTRACT

The instant invention relates to solutions and methods that can be used to present the user of an analyser with a test result without resorting to human blood or plasma and thereby overcoming the disadvantages of using blood or plasma each time an analyser is to be used. In some embodiments, a solution for use with one or more blood coagulation sensor substrates to generate a signal relating to the clot time is provided, wherein the solution is non-biohazardous.

### CROSS REFERENCE TO RELATED APPLICATIONS

This application claims the benefit of U.S. Provisional Application No. 62/410,565, filed Oct. 20, 2016, entitled NON-BIOHAZARDOUS SOLUTIONS AND METHODS FOR TESTING ANALYSERS. The entire contents of the foregoing are hereby incorporated by reference herein.

### BACKGROUND

In the detection and treatment of illness, it is useful to investigate properties of blood in patients. This is often done by taking a blood sample, such as a small sample from a finger stick, or larger sample from a venous or arterial draw. Blood can be analysed in a variety of analysers, including point of care devices. Point of care blood analysers are used by a variety of persons, such as medical professionals in hospital or clinical environments, as well as patients themselves undertaking a self-test. A blood sample is typically drawn from the patient and applied to the analyser to obtain a result. Such tests in coagulation include, but are not limited to, Activated Clotting Time (ACT), Activated Partial Thromboplastin Time (APTT), ProThrombin/International Normalized Ratio (PT/INR) tests to determine certain coagulation properties of the blood, and the like.

A typical ACT point of care test can involve applying blood to a test strip, where the test strip contains a contact activator such as kaolin clay or Celite®. The contact activator activates components in the blood to trigger the formation of a clot.

A typical PT/INR point of care test can involve applying blood to a test strip, where the test strip contains tissue factor to stimulate the formation of a blood clot. Different analysers can measure different aspects of the clot formation. For example, the Siemens Healthineers Xprecia Stride system measures the components formed by the reaction of the blood with the introduced tissue factor.

A variety of users need to correctly use and understand how a point of care analyser operates, and it is necessary to train each user in the operation of the analyser. Point of care analysers often include multiple steps to obtain a proper read of the properties of the blood sample, and may provide different results/outputs depending on the properties of the blood sample. Some users, particularly self-test patients, may be unfamiliar with the use of point of care analysers and technology in general. It may be necessary for the user to perform several operations of the analyser to become familiar with the procedures, outputs and user interface. Some point of care analysers, for example those testing coagulation properties, only respond to blood, or to a plasma sample derived from blood, where incorrect fluid or incorrect operation will result in an error.

It can therefore be difficult to train potential users in the operation of a point of care analyser, as it usually requires potentially biohazardous samples such as blood or plasma or liquid quality controls to provide a result. Sometimes multiple tests are required during training or a demonstration, and therefore multiple samples may be required. It may require several finger stick samples to be taken, causing multiple injuries to a person. In other situations, blood may be taken from a person other than the trainee or trainer, introducing the possibility of contamination, disease transmission, and the creation of biohazardous waste.

On other occasions, the people who perform the demonstration may not be trained to handle potentially biohazardous solutions. Examples include:

1) Presentations by executives to potential investors.

2) Public lectures by R&D scientists.

3) Demonstrations at clinical conferences by marketing specialists.

4) Deliberate generation/testing of errors by engineering staff.

5) Testing of new algorithms by software engineers.

6) Training of new users by salespeople.

7) Rapid assessment of returned sensors by technicians.

On yet other occasions, such as a university lecture theatre or a trade exhibit area in a conference, the area in which the blood coagulation sensor is to be demonstrated may not be set up for the handling of biohazardous samples, such as blood or liquid quality controls. Examples include. Liquid quality controls are also disadvantageous because they are expensive, lack stability, typically require reconstitution, which can be inconvenient, and comprise biohazardous material, such as plasma.

These disadvantages make the successful demonstration and training of the use of a point of care analyser potentially difficult or expensive, and/or introduce health risks.

Disclosed herein are solutions and methods to overcome some of the disadvantages of using blood or plasma in the demonstration, training or other non-diagnostic use of blood coagulation analysers such as point of care analysers.

### SUMMARY

As described herein, obtaining blood or plasma samples for non-diagnostic purposes can be difficult, and create hazardous waste.

The instant invention relates to solutions and methods that can be used to provide the user of an analyser with a test result without the use of human blood or plasma thereby overcoming the disadvantages of using blood or plasma each time an analyser is to be used.

In some embodiments, a solution disclosed herein is for use with one or more blood coagulation sensor substrates to generate a signal relating to the clot time is provided, wherein the solution is non-biohazardous. In some embodiments, the solution disclosed herein includes a proteolytic enzyme that cleaves a peptide whose C-terminus contains an amino acid linked to an electrochemical mediator or a colourimetric or fluorogenic reporting group. In some embodiments, the enzyme cleaves the carboxyl side of an arginine. In some embodiments, the amino acid can be linked to the electrochemical mediator or a colourimetric or fluorogenic reporting group via an amide bond.

In some embodiments of the invention, the proteolytic enzyme releases an inactive electrochemical mediator from the peptide to generate an active electrochemical mediator. In some embodiments, the active electrochemical mediator can be quantified by an electrochemical method. In some embodiments, the electrochemical method can chronoamperometry. In some embodiments, the proteolytic enzyme is a serine protease. The serine protease can be trypsin or thrombin.

In some embodiments of the invention disclosed herein, the solution can further include a buffer, a surface active species, and/or a stabiliser. In some embodiments, the solution can further include a component to overcome error checks in an analyser.

In one embodiment, a non-biohazardous stabilized solution is provided that can includes a protease that cleaves a thrombin substrate. For example, the protease can be trypsin or thrombin.

In some embodiments, the protease disclosed herein, such as trypsin, can be stabilised by a variety of methods, including low temperature, relatively high concentrations of divalent cations such as calcium or magnesium, reductive methylation of lysine residues, and/or lyophilisation of the protease followed by reconstitution with a separate liquid sample.

Also provided herein are support entities which can stabilize the protease in a solution. The support entities can include a stabilizing component to prevent autolysis of the protease. For example, in some embodiments, trypsin can be stabilised by calcium ions. In some embodiments, the support entities can include a pH buffer. Some analysers perform tests on a sample, for example to ensure the strip has filled correctly, not been damaged, and that the fluid deposited is blood. In some embodiments, the addition of elements such as a salt can allow the meter to accept the non-biohazardous sample that does not contain blood.

Some embodiments of the invention disclosed herein relate to a kit that includes one or more non-biohazardous solutions, and/or a kit that includes one or more solids and liquids to create one or more non-biohazardous solutions.

Some embodiments of the invention include methods for generating a clot time using a non-biohazardous solution disclosed herein with one or more blood coagulation sensor substrates. In some embodiments the methods can include mixing a non-biohazard solution disclosed herein with one or more blood coagulation sensor substrates linked to an electrochemical mediator, measuring the electrochemical mediator, and generating a clot time.

Some embodiments include methods for generating a non-biohazardous solution as disclosed herein for use with one or more blood coagulation sensor substrates in an analyser to generate a clot time. The methods can include mixing a pH buffer with a proteolytic enzyme, determining the concentration of the proteolytic enzyme based on timescale and threshold of the analyser, and generating a non-biohazardous solution for use with one or more blood coagulation sensor substrates in the analyser to generate a clot time.

Some embodiments of the invention relate to trypsin for use as a proteolytic enzyme in a non-biohazard solution to mimic thrombin in blood.

### BRIEF DESCRIPTION OF THE DRAWINGS

FIG. 1 is a schematic of the makeup of classical blood coagulation pathways.

FIGS. 2A and 2B are graphical representations of test results obtained with test solutions as described in Example 1.

FIG. 3 shows a graphical representation of stability data for a test solution.

FIGS. 4A and 4B are graphical representations of test results obtained with test solutions as described in Example 2.

FIGS. 5A and 5B are graphical representations of test results obtained with test solutions as described in Example 3.

### DETAILED DESCRIPTION

Disclosed herein is a non-biohazardous material that generates a response when applied to a test strip of an analyser. Some blood coagulation sensors contain a peptide linked to a reporter group. When the intrinsic or extrinsic blood clotting pathways are activated, the thrombin generated cleaves the reporter group off the peptide thus making the reporter group electrochemically active or changing its spectrophotometric characteristics. Application of a potential between two electrodes allows an electroactive reporter group to be sensed using chronoamperometry or other electrochemical techniques. Detection of changes in absorbance or other optical properties via detection of transmitted, reflected or fluorescent light allows a chromogenic or fluorogenic reporter group to be sensed.

Embodiments of the invention described herein relate to a non-biohazardous solution that can comprise a proteolytic enzyme. The proteolytic enzyme can be a serine protease. For example, the enzyme can be trypsin, or the like. The serine protease can cleave peptides on the carboxyl-side of arginine or lysine. Various concentrations of the serine protease can be used to generate a range of clot times depending on the analyzer. For example, the concentration can be based on timescales and detection details of the test for which the solution is formulated. A person of skill in the art would be able to use the teachings herein with respect to determining the concentration for the tests disclosed herein to determine the concentration for other tests. For example, if a particular concentration of proteolytic enzyme generates a clot time response that is too fast to demonstrate a particular type of blood coagulation test then the concentration of proteolytic enzyme can be decreased.

The proteolytic enzyme can be trypsin, bromelain (from pineapple), papain (from papaya, actinidin (from Kiwi fruit), ficin (from figs), recombinant factor Xa (cleaves next to arginine, recombinant thrombin, Pronase (from Streptomyces griseus), or the like. The enzyme can be lyophilized.

The invention described herein uses a proteolytic enzyme, such as trypsin, in a test solution to mimic the action of thrombin. This strategy works because both trypsin and thrombin are serine endopeptidases which cleave peptides on the carboxyl-side of arginine or lysine. Various concentrations of trypsin can be used to generate a range of clot times.

Trypsin can be isolated from various non-biohazardous invertebrate or vertebrate sources such as, crayfish, tunicates, lampreys, salmon, chickens, pigs, mice, and the like.

In some embodiments, the trypsin concentration can be chosen to emulate the range of INR values typically encountered in clinical samples. In other embodiments, trypsin concentrations which fall within the range of INR values expected for Liquid Quality Control (LQC) solutions employed by various analysers can be used.

The solutions and methods disclosed herein are designed to provide values which fall within the typical range of INR or ACT results expected in clinical situations. The APTT values reported herein are indicative of whole blood, one stage, uncalibrated APTT values. It will be apparent to one skilled in the art that adjustments can tune the apparent APTT values to fall within a different range.

The proteolytic enzyme can be stabilized by a variety of methods. For example, the enzyme can be stabilized with low temperature, relatively high concentrations of divalent cations such as calcium or magnesium, reductive methylation of lysine residues, and/or lyophilisation of the enzyme followed by reconstitution with a separate liquid sample.

In some embodiments the solutions disclosed herein have a shelf life of more than 1 week, 2 weeks, 3 weeks, 4 weeks, 5 weeks, 6 weeks, 12 weeks, 4 months, 6 months 1 year, 2 years, 3 years, 4 years, or more at temperatures of 4, 20, 30, or 40 degrees Celsius, and preferably about 4 degrees Celsius or about 20 degrees Celsius.

The non-biohazardous solution can further include a buffer, a surface active species, a stabilizer, or any combination thereof. The buffer, surface active species, and/or stabilizer that is used can be determined depending on the analyzer. The buffer can be Tris, MOPS, Hepes, PIPES, and the like The surface active species can be a detergent, preferably nonionic in nature, such as Tween-20, Triton X-100, Brij 35, Nonidet P40, and the like. The stabilizer can be calcium, magnesium, and the like. Concentrations of the buffer, surface active species, and/or stabilizer can be adjusted depending on the analyzer. In some embodiments, the invention does not require antimicrobial preservatives.

The non-biohazardous solution can further include a component to overcome error traps of an analyzer. For example, components, such as glycerol, dextrans or hydroxymethylcellulose, and the like, can be added to increase viscosity of the solution or components can be used to add or change the color of the solution. Further, salt can be added to adjust the ion content of the solution.

Embodiments of the invention relate to a kit including a premixed non-biohazardous solution in a bottle or vial. In other embodiments, the kit can include a first bottle or vial including the lyophilized proteolytic enzyme, and a second bottle or vial including a liquid. The solutions of the first and second vials can be mixed prior to use. The kit can further include instructions for using the solution.

Embodiments of the invention relate to methods of producing a non-biohazardous solution for use with one or more blood coagulation sensor substrates to generate a signal relating the clot time. The method can include mixing a pH buffer with a proteolytic enzyme and determining the concentration of the proteolytic enzyme based on timescale and threshold of an analyzer. In some embodiments, the selection of the pH can be guided by the optimal pH range for trypsin activity (pH 7-9).

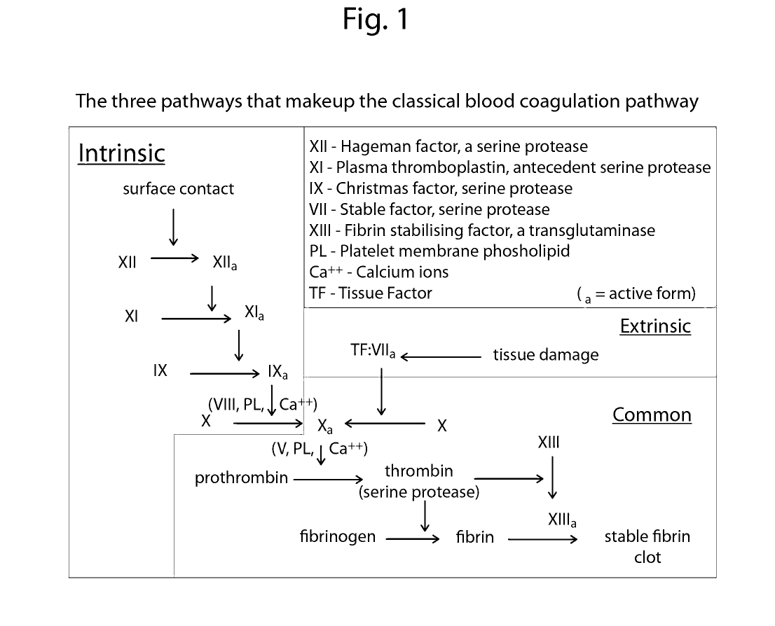

FIG. 1 shows the classical coagulation pathway for the formation of clots. Different tests performed on, for example, point of care analysers, attempt to measure the clotting time or ability of human patient blood. One test, such as for Activated Clotting Time (ACT), is performed by placing a human blood sample on a test strip, and measuring the intrinsic pathway through surface contact of the blood with an activator. A typical activator on the test strip may be kaolin clay. Application of blood to the test strip will activate the intrinsic pathway in the blood, causing a coagulation cascade generating a number of blood factors, which react with components of the blood or test strip such as phospholipids and calcium ions to form thrombin from prothrombin. Thrombin does not typically exist in appreciable concentrations in an uninjured healthy patient.

Another test is Prothrombin Time/International Normalized Ratio (PT/INR) which measures clotting through the extrinsic pathway as shown in FIG. 1. Here a blood sample can be applied to a test strip incorporating tissue factor. The blood sample interacting with the tissue factor simulates tissue damage, and generates factor IIa (thrombin).

In analysers such as the i-Stat hand-held blood analyser manufactured by Abbot Point of Care, thrombin levels generated in the test strip are measured to determine clotting ability of the blood sample. A thrombin substrate is cleaved by the thrombin formed in the sample; this process generates a leaving group. Thrombin cleaves peptide chains on the carboxyl side of the amino acid arginine. Various detection methods may be used to detect the cleaved leaving group, such as known colorimetric or electrochemical methods.

### EXAMPLES

### Example 1: UBI PT/INR Strips

In a first example, 0.5 M Tris, pH 7.5, 50 mM CaCl₂ and 1 mg/mL Tween-20 was spiked with various concentrations of trypsin (0.0625 ug/mL to 10 ug/mL). In this example porcine pancreatic trypsin was used.

The range of trypsin concentrations used was chosen to emulate the range of INR values typically encountered in clinical samples. However as described herein, in other uses, it may also be advantageous to use trypsin concentrations which fall within the range of INR values expected for Liquid Quality Control (LQC) solutions employed by various analysers.

The selection of the pH of 7.5 was guided by the optimal pH range for trypsin activity (pH 7-9). The relatively high concentration of calcium in this example was used to stabilise the trypsin against autolysis. The non-ionic detergent Tween-20 was included since it coats plastic surfaces and assists in preventing or slowing dilute proteins from sticking to the surfaces and denaturing.

The solution in this example is considered non-biohazardous. When tested with Siemens Healthineers Xprecia Stride strips and equivalent meters as liquid quality control solutions, the resulting transients were not trapped by the various error trap algorithms in the meters which are used to detect partial fills and exposed strips. Thus, the non-biohazardous solutions in this example were read by the meter as if they were liquid quality control samples. Typical liquid quality control solutions are bio-hazardous, being made from blood plasma, and have a short viable life once created.

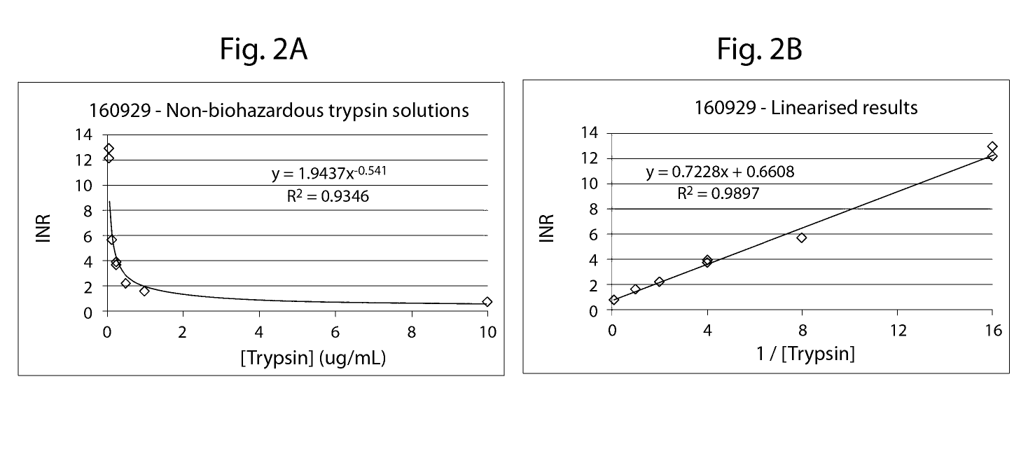

The range of INR values obtained by the “LQC algorithm” span the range that would be encountered in clinical samples (see FIG. 2A). A simple transformation of the X-axis linearises the data (see FIG. 2B). The values for FIGS. 2A and 2B are provided in Table 1 below.

TABLE 1

| [trypsin] | 1/trypsin | |
|---|---|---|
| (ug/mL) | (mL/ug) | INR |
| 10 | 0.1 | 0.72 |
| 10 | 0.1 | 0.73 |
| 1 | 1 | 1.56 |
| 1 | 1 | 1.56 |
| 0.0625 | 16 | 12.92 |
| 0.0625 | 16 | 12.16 |
| 0.125 | 8 | 5.64 |
| 0.125 | 8 | 5.67 |
| 0.25 | 4 | 3.70 |
| 0.25 | 4 | 3.90 |
| 0.5 | 2 | 2.14 |
| 0.5 | 2 | 2.19 |

Solutions containing 10 mM Tris, pH 7.5, 50 mM CaCl₂, 1 mg/mL Tween-20 and 0.1 mg/mL Indigo carmine (a blue food dye to help the user see the solution) were spiked with 0.87, 1.6 and 3.8 ug/mL TrypZean (a recombinant form of bovine pancreatic trypsin expressed in corn). The solutions were stored at −20, 4, 20, 30 and 40° C. and tested at various times over a 294 day period. A change in INR less than 0.5 units (for INR values less than 2) or 30% (for INR values greater than 2) was deemed acceptable.

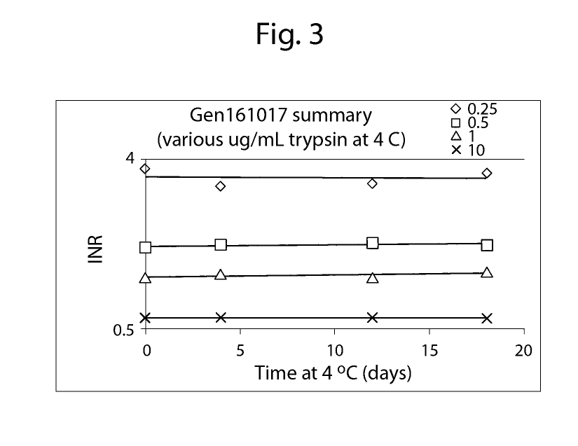

The results in FIG. 3 show that the 3.8 ug/mL trypsin solutions that were stored at 4 and 20° C. had results within the acceptable range over the entire trial period. The shelf life of the 3.8 ug/mL trypsin solution at various temperatures is summarised in the table below. The results show that the solution can survive extremes in temperature (several freeze-thaw cycles, or a few days at 40° C.). Moreover, it is possible to mix a solution as described above and store it at a refrigerated temperature for 4 years or at 20° C. for 1 year before use. In contrast, typical LQC samples must be used within 20 minutes of mixing.

TABLE 2

| Temperature | Shelf life | |
|---|---|---|
| (° C.) | (days) | Comments |
| −20 | | 9 freeze-thaw |
| | | cycles |
| 4 | 1648 | 4.5 years |
| 20 | 420 | 1.1 years |
| 30 | 85 | 12.1 weeks |
| 40 | 17 | 2.4 weeks |

The above example used relatively high calcium concentrations and relatively low temperature to prevent autolysis of trypsin. There are other ways of obtaining stable trypsin solutions including, for example:
1) Reductive methylation of the lysine residues in trypsin.
2) The use of specially designed recombinant trypsin molecules which have been optimised for stability. The other advantage of the recombinant approach is that the trypsin is not of animal origin and hence is highly unlikely to contain pathogenic organisms.

### Example 2: UBI ACT Strips

In a second example, a non-biohazardous test solution was created for an Intrinsic Pathway Assay 1 under development.

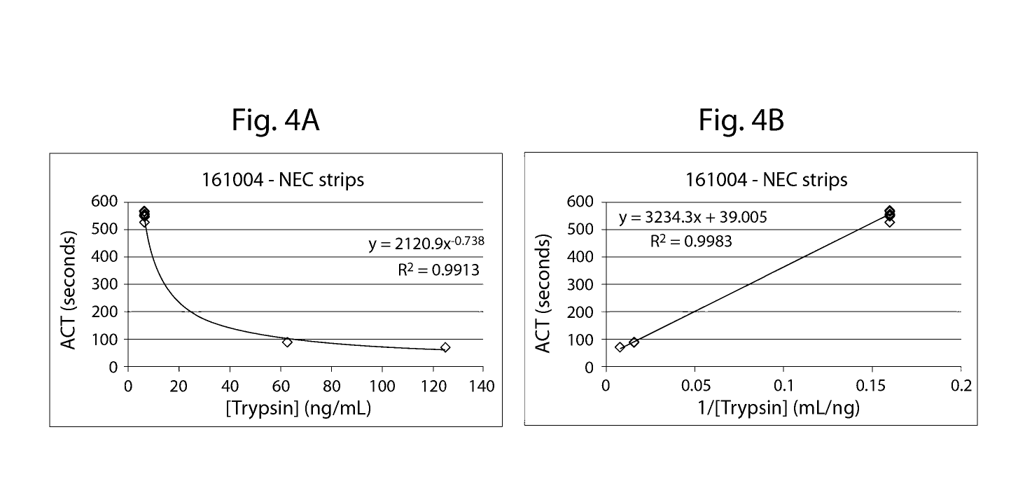

FIG. 4A shows a plot of the Intrinsic Pathway Assay 1 clot time versus various concentrations of trypsin (6.25 to 125 ng/mL), and FIG. 4B shows the linearised transformation. The values for FIGS. 4A and 4B are provided in Table 3 below.

TABLE 3

| [trypsin] | 1/[trypsin] | ACT |
|---|---|---|
| (ng/mL) | (mL/ng) | (s) |
| 6.25 | 0.16 | 567.75 |
| 6.25 | 0.16 | 567 |
| 6.25 | 0.16 | 571 |
| 6.25 | 0.16 | 525 |
| 6.25 | 0.16 | 546.75 |
| 6.25 | 0.16 | 557.75 |
| 6.25 | 0.16 | 554 |
| 6.25 | 0.16 | 563.75 |
| 6.25 | 0.16 | 550.75 |
| 6.25 | 0.16 | 568 |
| 6.25 | 0.16 | 550.5 |
| 62.5 | 0.016 | 84.75 |
| 62.5 | 0.016 | 88.25 |
| 62.5 | 0.016 | 87.25 |
| 62.5 | 0.016 | 89.5 |
| 62.5 | 0.016 | 89 |
| 125 | 0.008 | 68 |
| 125 | 0.008 | 69 |
| 125 | 0.008 | 68.25 |
| 125 | 0.008 | 68.5 |

### Example 3: UBI Activated Partial Thromboplastin Time Strips

The non-biohazardous test solutions are also useful for an Intrinsic Pathway Assay 2 under development, which uses a different contact-factor activator.

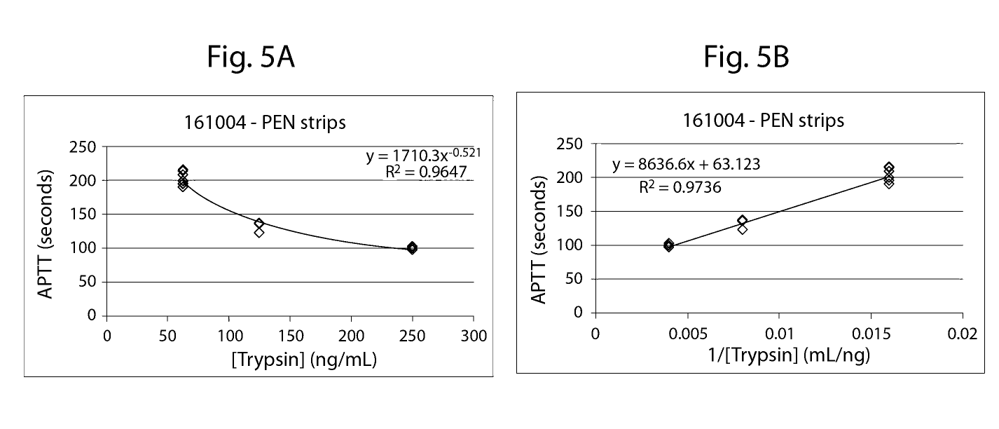

FIG. 5A shows a plot of the Intrinsic Pathway Assay 2 clot time versus various concentrations of trypsin (62.5 to 250 ng/mL), and FIG. 5B shows the linearised transformation. The values for FIGS. 5A and 5B are provided in Table 4 below.

TABLE 4

| [trypsin] | 1/[trypsin] | APTT |
|---|---|---|
| (ng/mL) | (mL/ng) | (seconds) |
| 62.5 | 0.016 | 201.5 |
| 62.5 | 0.016 | 200.5 |
| 62.5 | 0.016 | 189.75 |
| 62.5 | 0.016 | 183 |
| 62.5 | 0.016 | 197.25 |
| 62.5 | 0.016 | 180 |
| 62.5 | 0.016 | 182.25 |
| 62.5 | 0.016 | 183.25 |
| 125 | 0.008 | 105.75 |
| 125 | 0.008 | 121.75 |
| 125 | 0.008 | 120.5 |
| 125 | 0.008 | 110.75 |
| 250 | 0.004 | 83.25 |
| 250 | 0.004 | 82.75 |
| 250 | 0.004 | 87.5 |
| 250 | 0.004 | 80.75 |

### Example 4: Abbott i-Stat Meters and ACT Cartridges

It is possible to use the non-biohazardous solutions with i-Stat meters and ACT cartridges.

Table 5 below shows clot times obtained with different concentrations of trypsin (6.25 to 34.4 ng/mL).

TABLE 5

| [Trypsin] (ng/mL) | ACT (seconds) |
|---|---|
| 34.4 | 91 |
| 6.25 | 393 |

1. A solution for use with one or more blood coagulation sensor substrates to generate a signal relating to the clot time, wherein the solution is non-biohazardous.

2. The solution according to claim 1 comprising a proteolytic enzyme that cleaves a peptide whose C-terminus contains an amino acid linked to an electrochemical mediator or a colourimetric or fluorogenic reporting group.

3. The solution according to claim 2, wherein the enzyme cleaves the carboxyl side of an arginine.

4. The solution according to claim 2, wherein the amino acid is linked to the electrochemical mediator or a colourimetric or fluorogenic reporting group via an amide bond.

5. The solution according to claim 2, wherein the proteolytic enzyme releases an inactive electrochemical mediator from the peptide to generate an active electrochemical mediator.

6. The solution according to claim 5, wherein the active electrochemical mediator is quantified by an electrochemical method.

7. The solution according to claim 6, wherein the electrochemical method is chronoamperometry.

8. The solution according to claim 2, wherein the proteolytic enzyme is a serine protease.

9. The solution according to claim 8, wherein the serine protease is trypsin or thrombin.

10. The solution according to claim 2, further comprising a buffer, a surface active species and/or a stabiliser.

11. The solution according to claim 2, further comprising a component to overcome error checks in an analyser.

12. A kit comprising one or more non-biohazardous solutions, or solids and liquids to create one or more non-biohazardous solutions.

13. A method for generating a clot time using a non-biohazardous solution with one or more blood coagulation sensor substrates comprising:
mixing a non-biohazard solution with one or more blood coagulation sensor substrates linked to an electrochemical mediator;
measuring the electrochemical mediator; and
generating a clot time.

14.-15. (canceled)
